(19) United States
(12) Patent Application Publication
(10) Pub. No.: US 2026/0174376 A1
(43) Pub. Date: Jun. 25, 2026

(54) CEREBRAL VISION FUNCTION RAPID ASSESSING METHOD BASED ON ELECTROENCEPHALOGRAM AND FAST PERIODIC VISUAL STIMULATION ODDBALL PARADIGM, APPARATUS, AND STORAGE MEDIUM

(71) Applicant: SOUTH CHINA NORMAL UNIVERSITY, Guangdong (CN)

(72) Inventors: Juan CHEN, Guangdong (CN); Zhiqing DENG, Guangdong (CN); Pengmin QIN, Guangdong (CN)

(21) Appl. No.: 19/121,410

(22) PCT Filed: Dec. 26, 2023
(86) PCT No.: PCT/CN2023/142004

§ 371 Date: Apr. 15, 2025

(30) Foreign Application Priority Data
Feb. 13, 2023 (CN) 202310116864.9

(51) Int. Cl. A61B 5/378; A61B 5/00; A61B 5/374

(52) U.S. Cl. CPC A61B 5/378; A61B 5/374; A61B 5/7203; A61B 5/7246; A61B 5/7267

(57) ABSTRACT

A method for rapidly assessing cerebral visual function based on electroencephalogram (EEG) and fast periodic visual stimulation oddball paradigm includes: presenting several sets of visual stimuli images to a target individual and collecting EEG signals of the target individual while presenting the visual stimuli images, detecting a first and a second signal amplitude of the EEG signals, comparing the first signal amplitude with a third signal amplitude of a healthy individual in a database to determine a visual processing ability score of the target individual, and comparing the second signal amplitude with a fourth signal amplitude of the healthy individual in the database to determine a visual discrimination ability score of the target individual.

### CROSS-REFERENCE TO RELATED APPLICATIONS

This application is a national stage filing under 35 U.S.C. § 371 of international application No. PCT/CN2023/142004, filed Dec. 26, 2023, which claims priority to Chinese patent application No. 2023101168649 filed Feb. 13, 2023. The contents of these applications are incorporated herein by reference in their entirety.

### TECHNICAL FIELD

The present disclosure relates to the field of electroencephalogram (EEG) technologies, and in particular, to a method for rapidly assessing cerebral visual function based on EEG and fast periodic visual stimulation oddball paradigm, a computer apparatus, and a storage medium.

### BACKGROUND

Object recognition in human beings relies on the collection of visual information by eyeballs (similar to a camera) and the processing of incoming visual information by the brain (similar to a central processor). At this stage, optometrists and ophthalmologists have many ways to check whether the visual system of a patient's eyes is working properly. However, there are few approaches for assessing whether the brain effectively processes the visual information transmitted.

Studies have shown that after visual information is transmitted to the brain, it will be processed from low-level to high-level visual areas. Low-level visual information, such as contrast, orientation, spatial frequency, etc., can be processed in the primary visual cortex (V1), while middle-level features composed of low-level features, such as shape, contour, etc., need to be processed in the middle-level visual cortex such as V2, V3, and V4. Higher-level visual information, such as tools, faces, houses, natural scenes, etc., require processing in the high-level cortex, such as the lateral occipital lobe, ventral fusiform gyrus, ventral parahippocampal gyrus, and other brain regions. In addition, there are two different kinds of visual information: static and dynamic information. Static information is mainly processed in the ventral visual pathway, while dynamic visual information and motion-related information are mainly processed in the visual dorsal pathway. In other words, visual processing in the brain is in a fine and hierarchical way, from low-level features to high-level recognition.

There are specific special individuals, such as patients with disorders of consciousness (DOC), blindsight patients, and those with visual impairment caused by brain injuries, etc., whose ocular visual system may remain intact. Current tools for assessing cerebral visual abilities in such individuals include EEG-based Visual Evoked Potential (VEP) assessments and the visual subscale in the Coma Recovery Scale-Revised (CRS-R), which focuses on behavioral assessment.

VEP stimulates the retina with light flashes of a certain intensity to evaluate the visual information processing abilities by analyzing the latency and amplitude of P100 (a positive waveform occurring at about 100 ms after the stimuli) recorded in the occipital region. However, VEP mainly depends on the subjective interpretation of doctors and lacks objective comparison with the dataset of healthy controls. Moreover, VEP generally uses only a single visual stimulus type, making it challenging to generate a comprehensive perspective in hierarchical visual function assessment. The visual subscale of CRS-R uses specific instructions to assess whether patients can recognize objects, which requires special individuals to have a certain degree of understanding of the instructions. In addition, the behavioral assessment cannot fully reflect the cerebral visual ability of special individuals. In previous studies, functional magnetic resonance imaging (fMRI) technology has been used to assess visual function, but it requires people to complete visual tasks. Additionally, fMRI is expensive, time-consuming, and labor-intensive, requiring people to have no magnetically permeable substances in their bodies. Therefore, fMRI-based assessment methods are not suitable for special individuals.

A study has explored resting-state magnetic resonance imaging to assess the cerebral visual function of blind people, but this approach has not been applied to other individuals. Therefore, there is an urgent need to find a faster, more comprehensive, and accessible way to assess cerebral visual function that does not require language and motor abilities.

### SUMMARY

To address the technical challenges with existing methods for assessing human visual ability, which involve complex equipment, troublesome processes, fail to provide a comprehensive and hierarchical visual assessment, and other practical requirements, an objective of the present disclosure is to provide a method for rapidly assessing cerebral visual function based on EEG and fast periodic visual stimulation oddball paradigm, an apparatus, and a storage medium.

In accordance with an aspect of the present disclosure, an embodiment provides a method for rapidly assessing cerebral visual function based on EEG and fast periodic visual stimulation oddball paradigm, including:
presenting several sets of visual stimuli images to a target individual, with each set of visual stimuli images examining a specific visual function; wherein the several sets of visual stimuli images evaluate various cerebral visual functions of the target individual, from low-level to high-level processing; visual stimuli images in the same set include a series of base stimuli images and a series of oddball stimuli images; the base and oddball stimuli images are interleaved at specific frequencies to form a stimulus sequence; a base frequency is defined as a presentation frequency of the base stimuli images, and an oddball frequency is defined as a presentation frequency of the oddball stimuli images; the base frequency is greater than the oddball frequency, and the base frequency is a multiple of the oddball frequency;
collecting EEG signals of the target individual while presenting the visual stimuli images;
detecting the first and second signal amplitude of the EEG signals, where the first signal amplitude corresponds to the base frequency of the target individual, and the second signal amplitude corresponds to the oddball frequency and harmonics of the target individual.

Further, comparing the first signal amplitude with a third signal amplitude of a healthy individual in a database to determine a visual processing ability score of the target individual, and comparing the second signal amplitude with a fourth signal amplitude of the healthy individual in the database to determine a visual discrimination ability score of the target individual, including:
presenting several sets of visual stimuli images to the individuals in the healthy control group, where visual stimuli images in the same set include a series of base stimuli images and a series of oddball stimuli images, the base stimuli images and the oddball stimuli images in each set of visual stimuli images are interleaved at specific frequencies to form a stimulus sequence, a base frequency is defined as a presentation frequency of the base stimuli images, and an oddball frequency is defined as a presentation frequency of the oddball stimuli images;
collecting EEG signals of the individuals in the healthy control group while presenting the visual stimuli images;
detecting the third signal amplitude and the fourth signal amplitude of the EEG signals, where the third signal amplitude corresponds to the base frequency of the individuals in the healthy control group, and the fourth signal amplitude corresponds to an oddball frequency and harmonics of the individuals in the healthy control group;
determining Signal-to-Noise Ratios (SNRs) of the third signal amplitude and the fourth signal amplitude, respectively;
determining the visual processing ability score of the target individual according to a percentile of an SNR of the first signal amplitude in the SNR of the third signal amplitude; and determining the visual discrimination ability score of the target individual based on a percentile of an SNR of the second signal amplitude in the SNR of the fourth signal amplitude.

Further, several sets of base stimuli images and oddball stimuli images are hierarchically and systematically presented based on EEG and fast periodic visual stimulation oddball paradigm, where:
the base stimuli images are different from the oddball stimuli images at low-, middle-, and high-level visual functions;
at the low-level visual function, the base stimuli images and the oddball stimuli images have different contrasts/luminances, different sizes, different colors, different image orientations, or different spatial frequencies, respectively;
at the middle-level visual function, the base stimuli images and the oddball stimuli images have different image motion states (where base stimuli are randomly distributed points in a still state, and oddball stimuli are the same points in random motion) or different shapes, respectively; and
at the high-level visual function, the base stimuli images are randomly scrambled object images, and the oddball stimuli images are intact object images; or the base stimuli images are non-face object images, and the oddball stimuli images are face images; or the base stimuli images are non-tool object images, and the oddball stimuli images are tool images; or the base stimuli images are face images of strangers, and the oddball stimuli images are face images of familiar individuals; or the base stimuli images are face images of strangers, and the oddball stimuli images are self-face images.

Further, the base frequency is greater than the oddball frequency, and the base frequency is a multiple of the oddball frequency.

In accordance with another aspect of the present disclosure, an embodiment provides a computer apparatus, including a memory and a processor, where the memory is configured for storing at least one program. The processor is configured to load at least one program to execute the method for rapidly assessing cerebral visual function based on the EEG and fast periodic visual stimulation oddball paradigm in the above embodiment.

In accordance with another aspect of the present disclosure, an embodiment provides a storage medium having a processor-executable program stored therein. When executed by a processor, the processor-executable program causes the processor to implement the method for rapidly assessing cerebral visual function based on EEG and fast periodic visual stimulation oddball paradigm in the above embodiment.

The present disclosure has the following advantages. By rapidly assessing cerebral visual function based on EEG and fast periodic visual stimulation oddball paradigm according to the embodiment, base and oddball stimuli images are interleaved and presented to a target individual respectively at different set frequencies. EEG signals of the target individual are detected so that the visual processing ability and the visual discrimination ability of the target individual can be analyzed reliably and quantitatively. The method is implemented based on the EEG technique, requiring no provision of new equipment, efficient (i.e., can be completed within a few minutes), and suitable for assessing cerebral visual functions of various target individuals and has low requirements for the target individuals (where the target individuals are only required to view images with eyes, and the assessment can be finished without requiring language comprehension, or verbal and action response of the target individuals), can process data quickly (i.e., can provide an assessment result within a few minutes to ten-odd minutes), and has no negative impact on special target individuals.

### BRIEF DESCRIPTION OF DRAWINGS

FIG. 1 is a protocol of a trial of presenting base and oddball stimuli images under a tool condition in a fast periodic visual stimulation oddball paradigm according to an embodiment;

FIG. 2 is a schematic diagram of base and oddball stimuli images at different visual levels according to an embodiment;

FIG. 3 is a schematic diagram of a machine learning process according to an embodiment;

FIG. 4 is a schematic diagram of curves of the SNR spectrum that are obtained for healthy control groups in a fast periodic visual stimulation oddball paradigm according to an embodiment;

FIG. 5 is a schematic diagram of the SNR spectrum that is obtained in DoC groups in a fast periodic visual stimulation oddball paradigm according to an embodiment;

FIG. 6 is a schematic diagram of an integration result of the SNR of base frequency and averaged SNR of an oddball frequency and its harmonics for DoC groups and healthy control group according to an embodiment;

FIG. 7 is a schematic diagram showing a comparison result of SNRs of DoC groups and healthy control group according to an embodiment;

FIG. 8 is a schematic diagram showing a distribution of SNRs of base frequency in healthy control group according to an embodiment;

FIG. 9 is a schematic diagram showing a distribution of the averaged SNR of oddball frequency and its harmonics for healthy control group according to an embodiment; and

FIG. 10 is a flowchart of a method for rapidly assessing cerebral visual function based on EEG and fast periodic visual stimulation oddball paradigm according to an embodiment.

### DETAILED DESCRIPTION

An experiment is described first to assess cerebral visual functions in patients with cerebral visual impairment, providing a method for rapidly assessing cerebral visual function based on EEG and fast periodic visual stimulation oddball paradigm.

### (I) Image Stimuli

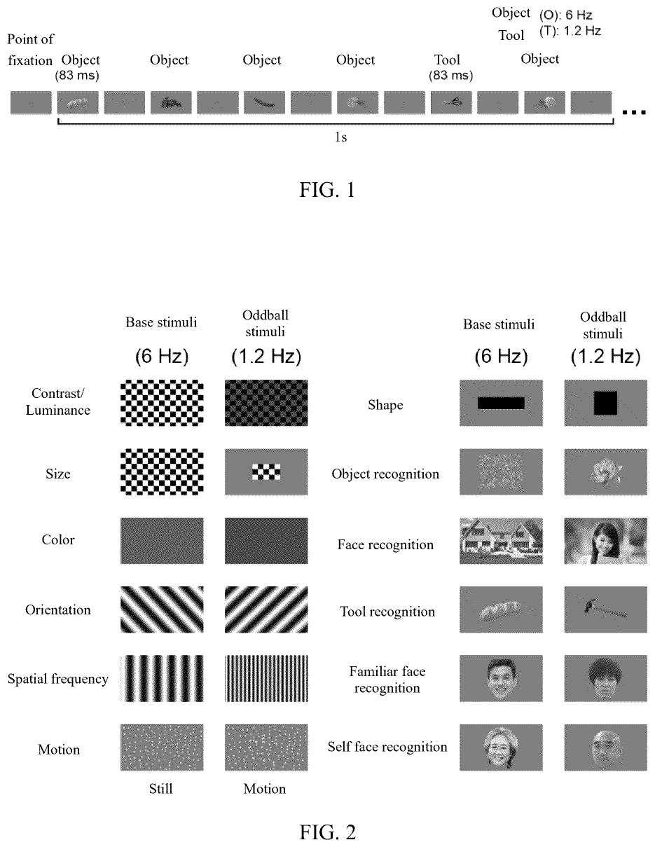

Combined with the electroencephalography technology, the fast periodic visual stimulation oddball paradigm presents a variety of visual stimuli from low to high levels to comprehensively and systematically assess the visual function in patients with DoC. Low-level visual conditions include contrast/luminance, size, color, orientation, and spatial frequency; middle-level visual conditions include motion and shape; and high-level visual conditions include object recognition, face recognition, tool recognition, familiar face recognition, and self-face recognition, as shown in FIG. 2. A base frequency is required to be a multiple of an oddball frequency, so that in this embodiment, the fast periodic visual stimulation oddball paradigm is realized with a base frequency of 6 Hz and an oddball frequency of 1.2 Hz. A tool condition is used as an example. FIG. 1 is a protocol of a trial under a tool recognition condition. Each condition includes two types of stimuli: base stimuli, with a total of 96 images presented at a frequency of 6 Hz, and oddball stimuli, with a total of 24 images presented at a frequency of 1.2 Hz. The visual stimuli in the paradigm will be described in detail below.

First: Contrast/luminance condition. The visual stimuli include black-and-white checkerboard images of only two different contrasts/luminances. All base stimuli images have one contrast/luminance value, and all oddball stimuli images have another contrast/luminance value.

Second: Size condition. The visual stimuli include black-and-white checkerboard images of two different sizes. The checkerboard area of the base stimuli images is larger, and the checkerboard area of the oddball stimuli images is smaller. The base and the oddball stimuli images are the same except for the size.

Third: Color condition. The colors of the base stimuli images and the oddball stimuli images are different. For example, the base stimuli images are of one color (such as red), and the oddball stimuli images are of another color (such as blue). The resolution of all the images is consistent. To eliminate the impact of the luminance factor, a luminance meter is used, and the images are set to the same luminance.

Fourth: Orientation condition. The black-and-white grating images oriented left and right are used as the base stimuli images and the oddball stimuli images, respectively. The spatial frequencies of the two types of raster images are kept consistent. The two types of images are the same except for the orientation.

Fifth: Spatial frequency condition. The spatial frequencies of the base stimuli images and the oddball stimuli images are different. The two types of images are the same except for the spatial frequency.

Sixth: Motion condition. Multiple white dots are presented on the screen. These white dots are in one of two states: static and moving. The dots in the static state are base stimuli, and the dots in the moving state are oddball stimuli.

Seventh: Shape condition. Shape patterns in the base stimuli images and the oddball stimuli images are different. For example, the shape in the base stimuli image is rectangular, and the shape in the oddball stimuli image is square.

Eighth: Object recognition condition. Colorful object images (24 images in total) and their corresponding randomly phase scrambled images (96 images in total, with consistent low-level visual information such as luminance and contrast) are presented on a gray background. At the center of each colorful object images (24 images) and within the pixel range of the object image, random scrambling is performed multiple times, to generate corresponding randomly scrambled images respectively. Four corresponding randomly scrambled images were generated for each colorful object image, i.e., a total of 96 randomly scrambled images were generated. Randomly phase scrambled images (96 images) were used as the base stimuli images, and complete colorful object images (24 images) were used as the oddball stimuli images. In this condition, the base frequency (6 Hz) reflects the ability to process randomly scrambled object images, and the oddball frequency (1.2 Hz) reflects the ability to discriminate between randomly scrambled images and intact object images.

Ninth: Face recognition condition. Ninety-six colorful non-face images were used as the base stimuli images, including fruits, plants, vegetables, desserts, musical instruments, furniture, animals, etc. Twenty-four colorful face images of individuals were used as the oddball stimuli images. The genders of the individuals in the images include male and female, and the ages of the individuals in the images cover children, teenagers, middle-aged people, and the elderly. The images were all from public galleries and have the same resolution. Under this condition, the base frequency (6 Hz) reflects the processing of object images, and the oddball frequency (1.2 Hz) reflects the ability to discriminate between face images and object images.

Tenth: Tool recognition condition. Ninety-six non-tool images were used as the base stimuli images, and 24 tool images were used as the oddball stimuli images, i.e., there are a total of 120 images, all of which are from a public gallery. To control the impact of shape, the two types of images are images of elongated objects with the same resolution. Under this condition, the base frequency (6 Hz) reflects the processing of non-tool object images, and the oddball frequency (1.2 Hz) reflects the ability to discriminate between tool object images and non-tool object images.

Eleventh: Familiar face condition. Ninety-six face images of strangers were used as the base stimuli images, and 24 face images familiar to the target individual were used as the oddball stimuli images. Under this condition, the base frequency (6 Hz) reflects the face processing, and the oddball frequency (1.2 Hz) reflects the ability to discriminate between familiar face images and unfamiliar face images.

Twelfth: Self-face condition. Ninety-six face images of strangers were used as the base stimuli images, and 24 face images of the target individual were used as the oddball stimuli images. Under this condition, the base frequency (6 Hz) reflects the face processing, and the oddball frequency (1.2 Hz) reflects the ability to discriminate between self-face images and unfamiliar face images.

### (II) Subjects

The subjects include 50 healthy subjects and 50 patients with DoC (24 patients in vegetative state (VS), 18 patients in Minimally Conscious State (MCS), and 8 patients in emergence from MCS (EMCS)).

### (III) Data Collection

During the EEG experiment, fast periodic visual stimulation paradigms under different conditions were randomly performed, and at the same time, the EEG signals of the participants were collected. Presenting visual stimuli of a “tool recognition” set is used as an example. In one second, four non-tool images (base stimuli images) are presented first. Then, one tool image (oddball stimuli image) is presented, followed by one non-tool image (base stimuli image). Each visual stimuli image, whether it is a base stimuli image or an oddball stimuli image, is presented for 83 ms. An interval between two visual stimuli images is 83 ms. See FIG. 1 for details. One trial lasts 20 seconds in total, and six trials are performed for each condition, i.e., each condition's fast periodic visual stimulation paradigm can be completed in two minutes. Therefore, 20 to 30 minutes are needed to complete the fast periodic visual stimulation paradigm with 12 conditions. In the EEG experiment, an external electrode placed at the tip of the participant's nose is used as a reference electrode. Electrode impedance was kept below 10 KQ. EEG was amplified with a gain of 1000 K, bandpass filtered at 0.05-100 Hz, and digitized at a sampling rate of 1000 Hz.

### (IV) Data Analysis

### 1. EEG Data Preprocessing

Under each condition, the preprocessing of EEG data collected from each trial includes baseline correction, low-pass filtering, and re-referencing. Under each condition, the preprocessed data of each valid trial were averaged. The following analysis is based on averaged event-related potential (ERP) data.

### 2. Spectrum Analysis

A Fast Fourier Transform (FFT) is performed on the averaged ERP data under each condition to obtain a spectrum. The same spectrum analysis was performed for each participant, and then spectral data of all participants under each condition were averaged.

### 3. SNR

Based on the result of spectrum analysis, the ratio between the amplitude at frequencies of interest and the average of the 20 surrounding bins (10 on each side, excluding the immediate adjacent bin) is calculated to obtain the SNRs of the frequencies of interest. This procedure was used to calculate the SNR of baseline frequency (6 Hz) and oddball frequency (1.2 Hz).

### 4. Bayesian Statistics

To calculate whether the SNRs of the base frequency (6 Hz) and the oddball frequency (1.2 Hz) of each group of subjects under each condition are significantly greater than 1, Bayesian t-test is used. The reason for using Bayesian statistics is that it is suitable for statistics of small samples. If a Bayes factor obtained by the Bayesian t-test is greater than 1 and less than 3, it indicates weak evidence to support the hypothesis that the SNR is significantly greater than 1. If the Bayes factor is greater than 3 and less than 10, it indicates moderate evidence to support the hypothesis that the SNR is significantly greater than 1. If the Bayes factor is greater than 10, it indicates strong evidence to support the hypothesis that the SNR is significantly greater than 1.

### 5. Correlation Analysis

To verify whether the consciousness level of patients with DoC can be detected by using the fast periodic visual stimulation oddball paradigm, we calculate the correlation between the SNRs of the base frequency (6 Hz) and the oddball frequency (1.2 Hz) and its harmonics (averaged among 1.2, 2.4, 3.6, 4.8, and 7.2 Hz) obtained under this paradigm and the visual subscale score and the total score of the CRS-R scale.

To compare the visual assessment technique based on the fast periodic visual stimulation paradigm with existing commonly used visual assessment techniques, we also computed the correlation between the base frequency and the oddball frequency obtained under this paradigm and VEP.

### 6. Machine Learning

A machine learning method is used to further explore the relationship between visual processing and discrimination abilities and consciousness level, and to find markers related to consciousness in vision.

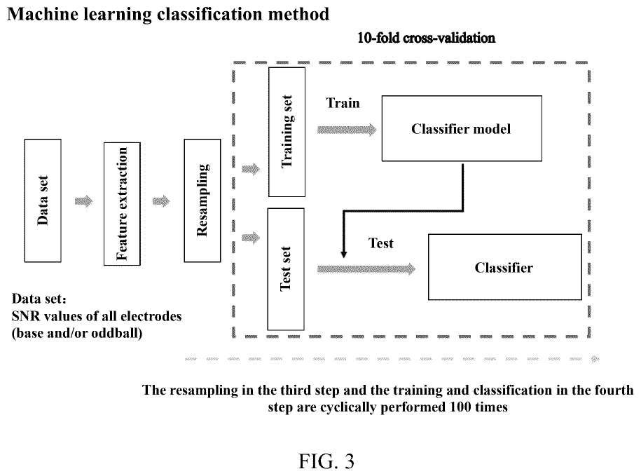

Referring to FIG. 3, a specific machine learning process is as follows.

In the first step, a dataset is constructed. The SNRs of the base frequency and the oddball frequency (and its harmonics) obtained under each condition in the fast periodic visual stimulation oddball paradigm are used as the dataset.

In the second step, feature extraction is performed. Only ten conditions are included here: contrast/luminance, size, color, orientation, spatial frequency, motion, shape, object recognition, face recognition, and tool recognition. In the feature extraction step, the base or oddball frequencies (or averaged oddball frequency and its harmonics) of a single condition or a combination of multiple conditions are extracted as features.

In the third step, resampling is performed to balance the sample size. Because different groups of subjects have different sample sizes, the sample sizes are unbalanced. Herein, the resampling method is used to balance the sample sizes.

In the fourth step, training and classification are performed. Based on the data after resampling, two-class classification is respectively performed among vegetative state, MCS group, EMCS patients, and healthy control group by using 10-fold cross-validation with Linear Discriminant Analysis (LDA), Support Vector Machine (SVM), K-nearest neighbor, neural network, or random forest as a two-class classifier. Similarly, three-class classification is performed among vegetative state, MCS, and healthy control group by using 10-fold cross-validation with an SVM three-class classifier. A classification accuracy reported in the 10-fold cross-validation is an average of the classification accuracies for ten times of training and classification. The purpose of using 10-fold cross-validation is to obtain an accurate and stable classification accuracy.

### (V) Results

### 1. Results of Healthy Control Group

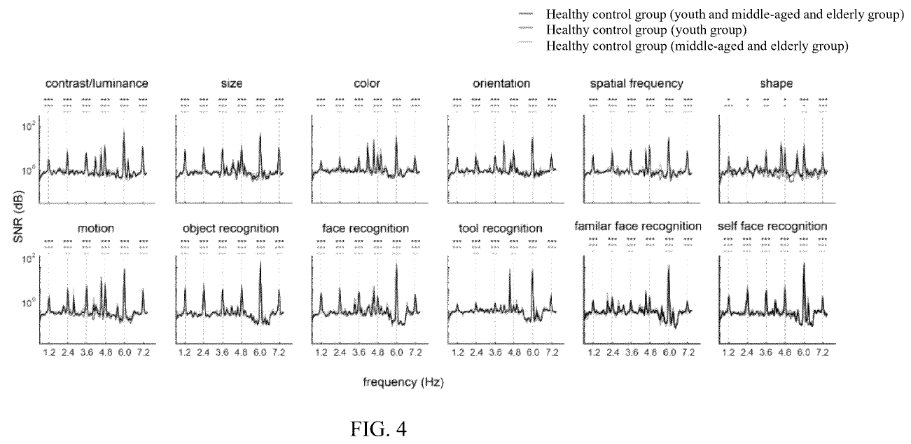

Bayesian t-test is performed in group analysis. The results show that under the conditions of contrast/luminance, size, color, orientation, spatial frequency, motion, shape, object recognition, face recognition, tool recognition, familiar face recognition, and self-face recognition, the SNRs of the base frequency and the oddball frequency obtained for the healthy control group in the fast periodic visual stimulation oddball paradigm are significantly greater than 1 (as shown in FIG. 4). This indicates that healthy subjects can process and discriminate visual stimuli under all conditions. This paradigm can effectively reflect the processing and discrimination abilities of healthy subjects.

Referring to FIG. 4, Bayesian t-test is used for each condition and each group to calculate whether the SNRs (dB) of the base frequency of 6 Hz and the oddball frequency of 1.2 Hz and its harmonics (2.4, 3.6, 4.8, and 7.2 Hz) are significantly greater than 1.

### 2. Results of DoC Group

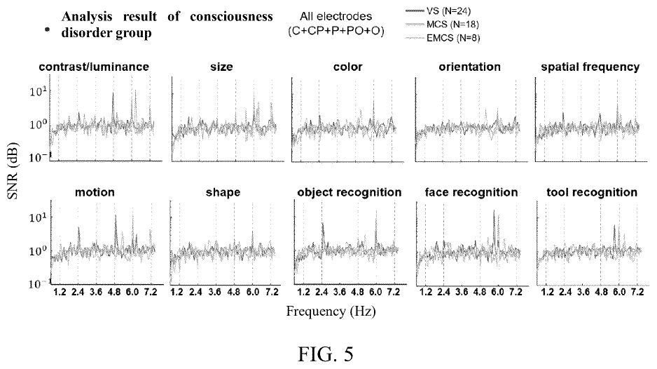

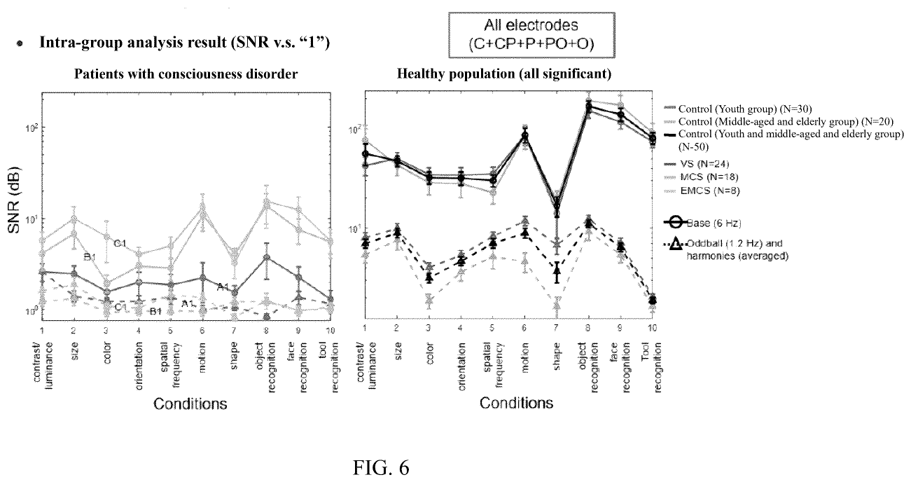

Because the number of subjects in the DoC group tested under the conditions of familiar face recognition and self-face recognition is too small, the following results only include ten conditions: contrast/luminance, size, color, orientation, spatial frequency, motion, shape, object recognition, face recognition, and tool recognition. Group analysis is performed using Bayesian t-test. The results show that patients in vegetative state only have the abilities to process (reflected in base frequency) and discriminate (reflected in oddball frequency and its harmonics) low-level visual stimuli (contrast, size, and color); and patients in MCS and EMCS states have almost all levels of visual processing abilities (reflected in base frequency), and part of low-level, middle-level, and high-level visual discrimination abilities (reflected in oddball frequency and its harmonics). See FIG. 5 and FIG. 6 for details.

Referring to FIG. 5, Bayesian t-test is used for each condition and each group to calculate whether the SNRs (dB) of the base frequency of 6 Hz and the oddball frequency of 1.2 Hz and its harmonics (2.4, 3.6, 4.8, and 7.2 Hz) are significantly greater than 1.

Referring to FIG. 6, the horizontal axis represents conditions, and the vertical axis represents SNR (dB). Bayesian t-test is used for each condition and each group to calculate whether the average SNR (dB) of the base frequency of 6 Hz and the oddball frequency of 1.2 Hz and its harmonics (2.4, 3.6, 4.8, and 7.2 Hz) is significantly greater than 1. Whether the average SNR is significantly greater than 1 is determined according to the Bayes factor. A curve denoted by a solid line with dots represents the SNR of the base frequency (6 Hz), and a curve denoted by a dashed line with triangles represents the oddball frequency and its harmonics (averaged, 1.2, 2.4, 3.6, 4.8, and 7.2 Hz). In FIG. 6, a curve denoted by “A1” represents patients in vegetative state, a curve denoted by “B1” represents patients in MCS, and a curve denoted by “C1” represents patients in EMCS.

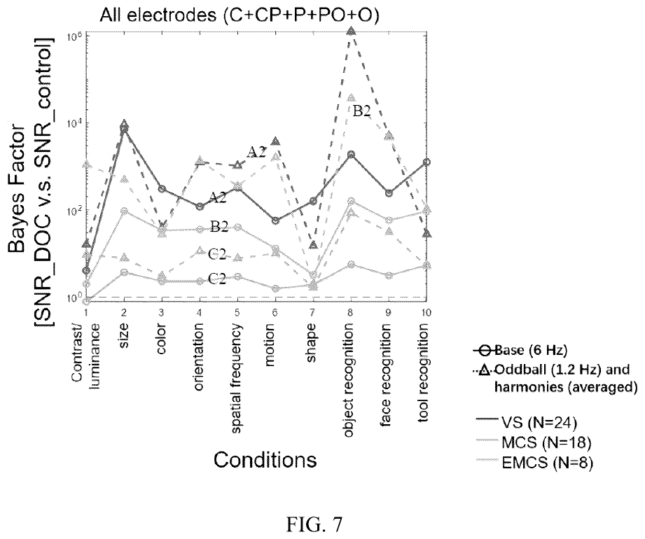

By comparing the SNR of the base frequency and the average SNR of the oddball frequency and its harmonics of the DoC group with those of the healthy control group, we found that the two groups were significantly different under all conditions. Moreover, the difference between the vegetative state group and the healthy control group is the largest, and the difference between the MCS group and the healthy control group is the second largest. The difference between the EMCS and healthy control groups is the smallest, as shown in FIG. 7.

Referring to FIG. 7, the horizontal axis represents conditions, and the vertical axis represents a Bayes factor obtained by performing Bayesian t-test on patients with DoC and the healthy control group. A curve denoted by a solid line with dots represents the SNR of the base frequency (6 Hz), and a curve denoted by a dashed line with triangles represents the oddball frequency and its harmonics (averaged, 1.2, 2.4, 3.6, 4.8, and 7.2 Hz). In FIG. 7, a curve denoted by “A2” represents patients in vegetative state, a curve denoted by “B2” represents patients in MCS, and a curve denoted by “C2” represents patients in EMCS.

### 3. Correlation Between SNR, CRS-R Score, and VEP

The SNRs of the base frequency, the averaged oddball frequency, and its harmonics (1.2, 2.4, 3.6, 4.8, and 7.2 Hz) of all patients with DoC under all conditions are respectively correlated with the visual subscale score and the total score of the CRS-R scale. The results show that the base frequency is significantly correlated to the visual subscale score of CRS-R almost under all conditions, and the oddball frequency is significantly correlated to the visual subscale score of CRS-R under only the object recognition condition, as shown in Table 1. This indicates that the visual subscale of CRS-R mainly reflects the ability to process visual information, and it did not perform well in terms of visual discrimination ability. The base frequencies of middle-level and high-level visual conditions are significantly related to the CRS-R total score, i.e., the base frequencies of middle-level and high-level visual conditions can also significantly reflect the consciousness level, as shown in Table 2.

The base frequencies (6 Hz) of all patients with DoC under all conditions are correlated with VEP's category results. The three conditions most correlated with VEP are size, motion, and object recognition conditions. The correlation between the classification result of VEP and the base frequency of the motion condition is the highest, at 0.53. The results show that the assessment based on VEP can only reflect the patient's visual processing ability to a certain extent and did not perform well in assessing the visual discrimination ability.

Based on the correlation between the SNRs obtained by this paradigm and the CRS-R score and VEP, it can be learned that CRS-R and VEP have shortcomings in assessing visual ability. CRS-R and VEP can only reflect the patient's visual processing ability, and cannot reflect the patient's visual discrimination ability.

TABLE 1
Correlation results between SNRs of the base frequency (6 Hz) and the oddball
frequency and its harmonics (oddball avg, averaged among 1.2, 2.4, 3.6,
4.8, and 7.2 Hz) under each condition and visual subscale scores of CRS-R

| Condition | SNR (Base) | SNR (oddball_avg) | SNR (base + oddball_avg) | Remark |
|---|---|---|---|---|
| 1-contrast/luminance | r = 0.23 | r = 0.15 | r = 0.25 | The table shows |
| 2-size | r = 0.32 | r = 0008 | r = 0.30 | the correlation |
| 3-color | r = 0.18 | r = −0.19 | r = 0.07 | between SNR |
| 4-orientation | r = 0.32 | r = −0.09 | r = 0.26 | values and visual |
| 5-spatial frequency | r = 0.37 | r = 0.05 | r = 0.34 | scores in CRS-R. |
| 6-motion | r = 0.41 | r = 0.06 | r = 0.39 | |
| 7-shape | r = 0.47 | r = −0.08 | r = 0.43 | |
| 8-object | r = 0.49 | r = 0.36 | r = 0.51 | |
| 9-face | r = 0.37 | r = −0.16 | r = 0.33 | |
| 10-tool | r = 0.46 | r = 0.06 | r = 0.46 | |

TABLE 2
Correlation results between SNRs of the base frequency (6 Hz) and the
oddball frequency and its harmonics (oddball avg, averaged among 1.2,
2.4, 3.6, 4.8, and 7.2 Hz) under each condition and CRS-R total score

| Condition | SNR (Base) | SNR (oddball_avg) | SNR (base + oddball_avg) | Remark |
|---|---|---|---|---|
| 1-contrast/luminance | r = 0.23 | r = 0.15 | r = 0.23 | The table shows |
| 2-size | r = 0.33 | r = 0.015 | r = 0.29 | the correlation |
| 3-color | r = 0.22 | r = −0.16 | r = 0.10 | between SNR |
| 4-orientation | r = 0.21 | r = −0.09 | r = 0.17 | values and the |
| 5-spatial frequency | r = 0.28 | r = 0.006 | r = 0.25 | CRS-R total score. |
| 6-motion | r = 0.42 | r = 0.12 | r = 0.39 | |
| 7-shape | r = 0.50 | r = −1.68e−04 | r = 0.49 | |
| 8-object | r = 0.35 | r = 0.36 | r = 0.39 | |
| 9-face | r = 0.31 | r = −0.23 | r = 0.25 | |
| 10-tool | r = 0.44 | r = 0.09 | r = 0.43 | |

### 4. Specific Applications

The multi-level fast periodic visual stimulation oddball paradigm can generate comprehensive visual ability reports for patients with DoC. On the one hand, according to a patient's base frequency and oddball frequency, it can be assessed whether the patient retained visual processing ability (whether the patient can receive visual signals) and discrimination ability (whether the patient can distinguish visual features). Specifically, the visual processing ability and visual discrimination ability of the patient under each condition are accessed depending on whether Z-scores of the base frequency and the oddball frequency obtained by a single electrode from the current patient under each condition are significantly greater than 1.64, to form a systematic visual ability assessment report rapidly. Table 3 shows the visual assessment report of one patient.

TABLE 3
Visual assessment report of a patient

| | | Can the patient | |
|---|---|---|---|
| | Can the patient | discriminate | |
| Assessment | receive visual | between visual | |
| item | signals (6 Hz) | features (1.2 Hz) | Remark |
| Visual contrast | Yes | Yes | |
| Size | Yes | Yes | |
| Color | Yes | Yes | A significant 1.2 Hz |
| | | | peak was observed |
| | | | only at P2 |
| Orientation | Yes | No | |
| Spatial | Yes | Yes | |
| frequency | | | |
| Motion | Yes | Yes | |
| Shape | Yes | Yes | |
| Object | Yes | Yes | |
| Face | Yes | Yes | |
| Tool | Yes | Yes | A significant 1.2 Hz |
| recognition | | | peak was observed |
| | | | only at CPZ and P4 |

Conclusions: The significant peak values at 6 Hz were observed under visual contrast, size, color, orientation, spatial frequency, motion, shape, object recognition, face recognition, and tool recognition conditions, indicating that the patient's brain could receive visual input. The significant peak values at 1.2 Hz were observed under visual contrast, size, color, spatial frequency, motion, shape, object recognition, face recognition, and tool recognition conditions. There was no significant peak value at 1.2 Hz in the orientation condition, indicating that the patient could distinguish visual stimuli. Further observation and assessment are still needed.

On the other hand, the patient's base frequency and oddball frequency may be compared with those of the healthy control group to form an objective visual processing ability and discrimination ability score report showing the comparison.

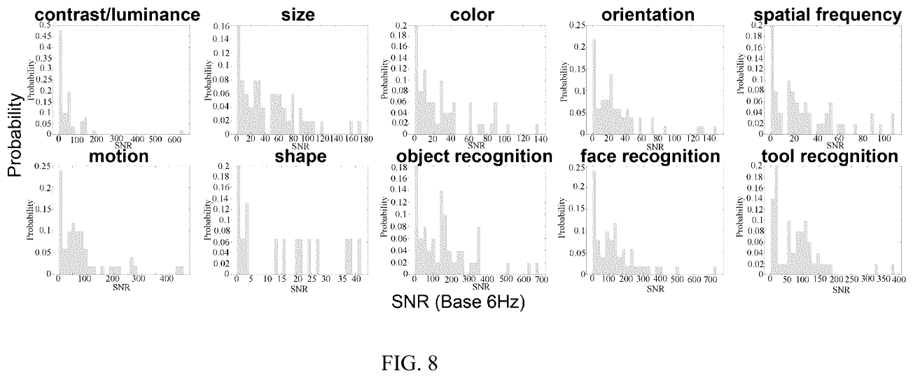

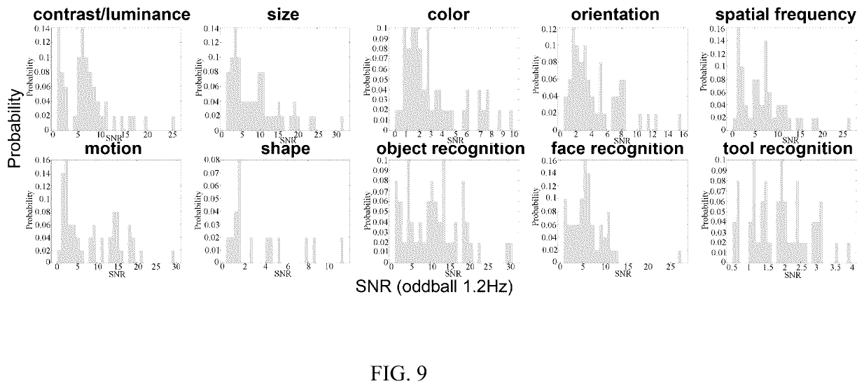

Conventional technologies, such as the CRS-R scale and VEP, have certain limitations. The visual subscale in the CRS-R scale requires patients to understand instructions, which may lead to misdiagnosis in patients with auditory deficiencies. VEP technology relies only on an individual's evoked potentials to assess visual reception ability, making it dependent on subjective interpretation by clinicians and lacking the use of a large dataset as a reference. To address this, the fast periodic visual stimulation oddball paradigm was also applied in the healthy control group, and a large dataset of the healthy control group was obtained, as shown in FIG. 8 and FIG. 9.

FIG. 8 shows a distribution of SNRs of a base frequency for healthy control group in contrast/luminance, size, color, orientation, spatial frequency, motion, shape, object recognition, face recognition, and tool recognition conditions. The horizontal axis represents the SNR at the base frequency of 6 Hz, and the vertical axis represents the probability of the SNR.

FIG. 9 shows a distribution of an averaged SNR of an oddball frequency and its harmonics for healthy control group in contrast/luminance, size, color, orientation, spatial frequency, motion, shape, object recognition, face recognition, and tool recognition conditions. The horizontal axis represents the SNR at the oddball frequency of 1.2 Hz, and the vertical axis represents the probability of the SNR.

Therefore, by comparing the base frequency and the oddball frequency and its harmonics of the DoC group with those of the healthy control group under the fast periodic visual stimulation oddball paradigm, results reveal the low-level, middle-level, and high-level visual processing and discrimination abilities of patients with DoC objectively. According to percentiles of SNRs of the base frequency and the oddball frequency and its harmonics of each patient under multiple conditions in an SNR of the healthy control group, a visual processing ability score and a visual discrimination ability score of the patient at each visual level can be obtained respectively (with a maximum score of 100 points). For example, patient P34 in Table 4 was in vegetative state. The percentiles of the SNRs for the base frequency and the oddball frequency and its harmonics under ten conditions were calculated, respectively, allowing us to obtain visual processing and discrimination scores of the patient under ten conditions. Then, the averaged percentiles of the SNRs of the base frequency across ten conditions were computed to obtain a visual processing ability score of 4.8 points (out of 100 points). Finally, the percentiles of the SNRs of the oddball frequency and its harmonics under the ten conditions were averaged to obtain an average visual discrimination ability score, which was 7.4 points (out of 100 points). Patient P57 was a patient in MCS. According to the same algorithm, the visual processing ability scores of patient P57 under the ten conditions were calculated, resulting in an average visual processing ability score of patient P57 of 12 points and an average visual discrimination ability score of patient P57 of 6.8 points. The data for patient P57 is in Table 4.

TABLE 4
Visual processing ability scores and visual discrimination ability scores of patient P34
in vegetative state and patient P57 in MCS under ten conditions, and their averages

| | Patient No. | | | |
|---|---|---|---|---|
| | P34 | | P57 | |
| | Patient type | | | |
| | | | MCS | |
| | Vegetative state | | | Visual |
| | | Visual discrimination | | discrimination |
| | Visual processing | ability score (oddball | Visual processing | ability score |
| | ability score (base | frequency and its | ability score (base | (oddball frequency |
| | frequency) | harmonics) | frequency) | and its harmonics) |
| Conditions | [out of 100 points] | [out of 100 points] | [out of 100 points] | [symbol] |
| Contrast/ | 12 | 4 | 12 | 14 |
| Luminance | | | | |
| Size | 6 | 0 | 8 | 0 |
| Color | 6 | 26 | 4 | 4 |
| Orientation | 6 | 10 | 14 | 10 |
| Spatial | 6 | 0 | 10 | 8 |
| frequency | | | | |
| Motion | 0 | 0 | 2 | 0 |
| Shape | 0 | 12 | 40 | 4 |
| Object | 0 | 0 | 12 | 0 |
| recognition | | | | |
| Face | 8 | 10 | 8 | 10 |
| recognition | | | | |
| Tool | 4 | 12 | 10 | 18 |
| recognition | | | | |
| Average | 4.8 | 7.4 | 12 | 6.8 |
| score | | | | |

[symbol]

indicates data missing or illegible when filed

### 5. Advanced Applications

The multi-level fast periodic visual stimulation oddball paradigm could be used in combination with a machine learning method (see FIG. 3 for the detailed process) to further explore the relationship between visual processing and discrimination abilities and consciousness level, as well as to identify vision-related markers of consciousness.

The two-class classification was performed among VS, an MCS, an EMCS group, and a healthy control group through machine learning with the base frequency and/or oddball frequency (and its harmonics) as features. When sample sizes were balanced, classification accuracy was high in motion, shape, and object recognition conditions. In summary:
(1) Distinguishing between VS and MCS groups, the classification accuracy reached 84% with SNRs of the base frequency (6 Hz) under the shape and tool conditions as features.
(2) Distinguishing between VS and EMCS groups, with the SNRs of the base frequency (6 Hz) and the oddball frequency (1.2 Hz) under most middle-level and high-level conditions as features, the classification accuracy was generally exceeding 70%, with a maximum of 79%.
(3) Distinguishing between MCS and EMCS groups, the classification accuracy reached 79% with the SNR of the oddball frequency (1.2 Hz) under the shape condition as features.
(4) Distinguishing between VS and healthy control groups, the classification accuracy reached 85% with the averaged SNRs of the base frequency and the oddball frequency and its harmonics under the shape condition as features.
(5) Distinguishing between MCS and healthy control groups, the classification accuracy reached 90% with the SNRs of the oddball frequency and its harmonics under the motion and shape conditions as features.
(6) Distinguishing between EMCS and healthy control groups, the classification accuracy reached 84% with the SNRs of the base frequency and the oddball frequency and its harmonics under the face recognition condition as features.

Three-class classification was performed for the VS, MCS, and healthy control groups. When sample sizes were balanced, the classification accuracy reached 75% with the averaged SNRs of the oddball frequency and its harmonics (averaged 1.2, 2.4, 3.6, 4.8, and 7.2 Hz) under the object recognition condition as features.

To sum up, the multi-level fast periodic visual stimulation oddball paradigm can efficiently and rapidly assess visual processing and discrimination abilities in patients with DoC. In addition, cerebral visual function is significantly related to consciousness under this paradigm. When distinguishing different types of patients, classification accuracy reached 84% with middle-level and high-level visual features.

Based on the above experiment, the following conclusions can be drawn. The fast periodic visual stimulation oddball paradigm enables rapid data processing. After preprocessing, the EEG data is subjected only to simple spectrum analysis and SNR calculation to obtain a patient's visual processing ability and discrimination ability for each visual level. This allows for forming a visual assessment report, providing additional guidance for patient recovery. In addition, machine learning can be used under the fast periodic visual stimulation oddball paradigm to further explore the relationship between visual processing and discrimination abilities and consciousness, as well as to identify potential markers of consciousness.

According to the conclusions of the above embodiment, this embodiment outlines a method for rapidly assessing cerebral visual function based on EEG and fast periodic visual stimulation oddball paradigm. The technique follows these principles. Images are presented to a patient, while EEG signals are collected to evaluate the cerebral visual function. The assessment may be performed with other EEG assessments in the hospital without requiring a separate EEG setup. The images are selected carefully based on the hierarchical structure of human visual processing, ranging from simple to complex. The images contain low-level visual information such as contrast, size, color, orientation, and spatial frequency, middle-level visual information such as motion and shape, and high-level visual information such as object recognition, face recognition, and tool recognition. There is also a condition to examine whether the patient can recognize his or her own faces and familiar faces. Each condition only takes two minutes for assessment. If the data analysis result shows that the patient retained low-level visual processing, further tests are performed to assess their ability to process higher-level information. If the data analysis result shows that the patient did not retain low-level visual function, the assessment will be terminated. In other words, the patient only needs to keep the eyes open without performing any other tasks, and the entire assessment can be completed within 2 to 30 minutes. The images are presented at a base frequency (e.g., 6 Hz), with some images periodically replaced by others presented at an oddball frequency (e.g., 1.2 Hz). If spectrum analysis of the EEG reveals a significant peak at the base frequency (6 Hz), the patient's visual input function is intact. On the contrary, if no peak at base frequency (6 Hz) is observed, it indicates impaired visual processing. If a significant peak at 6 Hz is observed but no 1.2 Hz peak is observed, it indicates that the patient has visual input, but cannot discriminate visual information at a certain level. If both significant peaks at base frequency (6 Hz) and oddball frequency (1.2 Hz) are observed, it indicates that the patient can discriminate between the images presented at 6 Hz and 1.2 Hz, suggesting that the patient retained relatively normal visual processing and discrimination abilities.

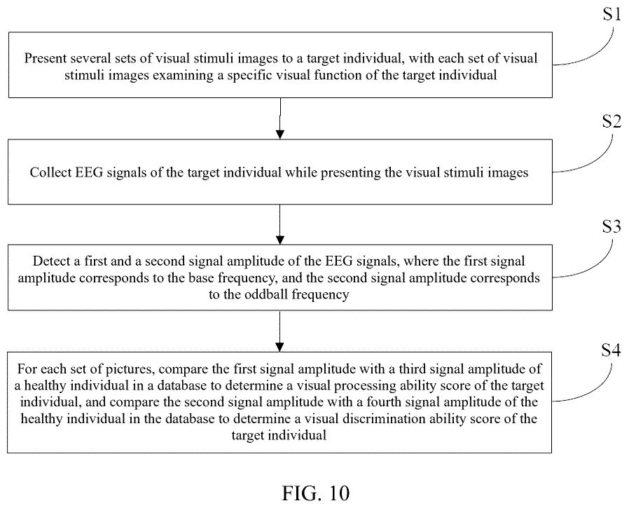

Based on the above principle, this embodiment, as illustrated in FIG. 10, describes a method for rapidly assessing cerebral visual function using EEG and fast periodic visual stimulation oddball paradigm. The method includes the following steps S1 to S4.

At S1, several sets of visual stimuli images are presented to a target individual, with each set of visual stimuli images examining a specific visual function. The several sets of visual stimuli images evaluate various cerebral visual functions of the target individual, from low-level to high-level processing.

Visual stimuli images in the same set include a series of base stimuli images and a series of oddball stimuli images. The base and oddball stimuli images are interleaved at specific frequencies to form a stimulus sequence. A base frequency is defined as a presentation frequency of the base stimuli images, and an oddball frequency is defined as a presentation frequency of the oddball stimuli images.

At S2, EEG signals of the target individual are collected while presenting the visual stimuli images.

At S3, a first signal amplitude and a second signal amplitude of the EEG signals are detected, where the first signal amplitude corresponds to the base frequency of the target individual, and the second signal amplitude corresponds to the oddball frequency and harmonics of the target individual.

At S4, the first signal amplitude is compared with a third signal amplitude of a healthy individual in a database to determine a visual processing ability score of the target individual, and the second signal amplitude is compared with a fourth signal amplitude of the healthy individual in the database to determine a visual discrimination ability score of the target individual.

In this embodiment, the steps of the method for rapidly assessing cerebral visual function based on EEG and fast periodic visual stimulation oddball paradigm can be executed by a computer device, referred to as a system for rapidly assessing cerebral visual function based on EEG and fast periodic visual stimulation oddball paradigm. This system includes four modules: the first module, the second module, the third module, and a fourth module configured for executing steps S1, S2, S3, and S4, respectively.

In S1, several sets of visual stimuli images are presented to a target individual, with each set of visual stimuli images examining a specific visual function. The several sets of visual stimuli images evaluate various cerebral visual functions of the target individual, from low-level to high-level processing. Visual stimuli images in the same set include a series of base stimuli images and a series of oddball stimuli images. The base and oddball stimuli images are interleaved at specific frequencies to form a stimulus sequence. A base frequency is defined as a presentation frequency of the base stimuli images, and an oddball frequency is defined as a presentation frequency of the oddball stimuli images.

In this embodiment, as illustrated in FIG. 2, a total of 12 sets of visual stimuli images are shown. Each set consists of base and oddball stimuli images, differentiated in contrast/luminance, size, color, orientation, spatial frequency, motion, shape, object recognition, face recognition, tool recognition, familiar face recognition, and self-face recognition.

In S1, the base and oddball stimuli images are interleaved at specific frequencies to form a stimulus sequence. The base frequency (e.g., 6 Hz) is defined as the presentation frequency of base stimuli images, and the oddball frequency (e.g., 1.2 Hz) is defined as the presentation frequency of the oddball stimuli images.

For example, in one trial (base stimuli images and oddball stimuli images of one set of visual stimuli images may be presented in each trial), the base stimuli images and the oddball stimuli images are alternately presented respectively according to set frequencies. A presentation frequency of the base stimuli images is 6 Hz, and a presentation frequency of the oddball stimuli images is 1.2 Hz.

As illustrated in FIG. 1, presenting visual stimuli images from “tool recognition” condition serves as an example. Within one second, four non-tool images (base stimuli images) are presented first, followed by one tool image (oddball stimuli image), and then one non-tool image (base stimuli image). Each visual stimuli image, whether base or oddball, is presented for 83 ms. An interval between two visual stimuli images is 83 ms.

For a target individual with DoC, six trials (two minutes) are performed for each level (contrast/luminance, size, color, orientation, spatial frequency, motion, shape, object recognition, face recognition, tool recognition, familiar face recognition, and self-face recognition). Therefore, the total duration for presenting all 12 sets of visual stimuli images is 20 to 30 minutes. The order of image sets is randomized.

In this embodiment, in S2, the EEG signals of the target individual are collected while executing S1, ensuring that the EEG data corresponds to the individual viewing the visual stimuli images.

In S3, the collected EEG signals of the target individual are preprocessed and undergo Fourier transformation, as well as other analytical methods to detect frequency components. The first signal amplitude is measured by detecting the EEG component corresponding to the base frequency (6 Hz) and the second signal amplitude is measured by detecting the EEG component corresponding to the oddball frequency (1.2 Hz).

In S4, for each image set, the first signal amplitude of the target individual is compared with a third signal amplitude from a healthy control database to determine the target individual's visual processing ability score. The second signal amplitude of the target individual is compared with a fourth signal amplitude from a healthy control database to determine the target individual's visual discrimination ability score.

Specifically, during or before the execution of S1, multiple sets of visual stimuli images are presented to the healthy control group (who are known to have no disease such as DoC) according to the same procedure as S1. EEG signals of healthy control group are collected while presenting the visual stimuli images. The EEG signals of the healthy control group are preprocessed, and undergo Fourier transformation, as well as other analytical methods. The third signal amplitude is measured by detecting the EEG component corresponding to the base frequency (6 Hz) for each of the individuals in the healthy control group, and an SNR may be calculated according to the third signal amplitude, thus obtaining an SNR of the third signal amplitude corresponding to each individual in the healthy control group. Similarly, the fourth signal amplitude is measured by detecting the EEG component corresponding to the base frequency (1.2 Hz) for each individual in the healthy control group. An SNR is calculated based on the fourth signal amplitude, yielding the SNR of the fourth signal amplitude corresponding to each individual in the healthy control group.

In S4, an SNR of the first signal amplitude is calculated. The SNRs of the third signal amplitudes obtained from the healthy control group form an SNR dataset. A percentile (from high to low) of the SNR of the first signal amplitude in the SNR dataset can be calculated. A visual processing ability score of the target individual (patient) is determined according to the percentile. For example, the percentile can be used directly as the visual processing ability score of the target individual (patient). Similarly, an SNR of the second signal amplitude is calculated. The SNRs of the fourth signal amplitudes obtained from the healthy control group form an SNR dataset. A percentile (from high to low) of the SNR of the second signal amplitude in the SNR dataset can be calculated. A visual discrimination ability score of the target individual (patient) is determined according to the percentile. For example, the percentile can be directly used as the visual discrimination ability score of the target individual (patient).

In S4, the third and fourth signals amplitude data of the individuals in the healthy control group are stored in a database, which is updated continuously. If the target individual is healthy, the first signal amplitude of the target individual will be added to the database as the new third signal amplitude data in the database, and the second signal amplitude will be added to the database as the new fourth signal amplitude data in the database. If the target individual is not healthy, the first and the second signal amplitude are separately stored as special individual data.

The experimental principle in this embodiment is applied in steps S1 to S4. For each set of images, the first signal amplitude of the target individual is compared with a third signal amplitude from the existing database to determine the target individual's visual processing ability score, and the second signal amplitude of the target individual is compared with a fourth signal amplitude from the existing database to determine the target individual's visual discrimination ability score.

In this embodiment, by using the electroencephalography technology commonly used in hospitals, the assessment of the cerebral visual function can be implemented by adding steps S1 to S4 to a routine EEG recording process for patients. Even for a patient with DoC who is unable to communicate with the external world and whose brain is largely unknown, steps S1 to S4 leverage the residual functions of the damaged brain. For example, patients in vegetative state still retain low-level visual processing and discrimination abilities, while those in MCS or EMCS retain most of the visual processing abilities and part of low-level, middle-level, and high-level visual discrimination abilities. These residual functions can serve as a key to facilitating patients' recovery. The method, including steps S1 to S4, is rapid and effective, enabling cerebral visual function assessment without requiring the patient to perform any task. The patient only needs to keep the eyes open to look at a display, without the need for any response. In addition, the assessment is safe and can be applied even if the patient has magnetically permeable implants in their body.

Specifically, based on the fast periodic visual stimulation oddball paradigm, the patient's visual function can be comprehensively and systematically assessed from low, middle, and high levels. For example, the low-level visual function of the patient can be assessed using the sets of base stimuli images and oddball stimuli images corresponding to visual features such as “contrast/luminance”, “size”, “color”, “orientation”, and “spatial frequency”. The middle-level visual function of the patient can be assessed using the sets of base stimuli images and oddball stimuli images corresponding to visual features such as “motion” and “shape”. The high-level visual function of the patient can be assessed using the sets of base stimuli images and oddball stimuli images corresponding to visual features such as “object recognition”, “face recognition”, “tool recognition”, “familiar face recognition”, and “self-face recognition”. Therefore, the EEG-based method for rapidly assessing cerebral visual function based on fast periodic visual stimulation oddball paradigm in this embodiment has the following advantages.
1. The method is based on EEG technology without requiring new equipment.
2. The method is efficient and suitable for patient's visual assessment.
3. The method has minimal requirements for patients and, therefore, allows for broad applications.
4. The method enables rapid data processing and provides results of the healthy control group as a reference.
5. The method is efficient, comprehensive, and suitable for visual assessment in patients.

A computer program for executing the method for rapidly assessing cerebral visual function based on EEG and fast periodic visual stimulation oddball paradigm in this embodiment may be written into a computer apparatus or a storage medium. When being read and executed, the computer program performs the method, thus achieving the same technical effects as described method for rapidly assessing cerebral visual function based on EEG and fast periodic visual stimulation oddball paradigm in this embodiment.

It should be noted that unless otherwise specified, when a feature is described as being “fixed” or “connected” to another feature, it may be directly or indirectly fixed or connected to another feature. In addition, as used in the present disclosure, the orientation or positional relationships indicated by the terms such as up, down, left, and right are based on orientation or position relationships between the components of the present disclosure shown in the accompanying drawings. As used in the present disclosure, the singular forms “a,” “an,” and “the” are intended to include the plural forms as well, unless the context clearly dictates otherwise. Furthermore, unless otherwise defined, the meanings of all technical and scientific terms used in this specification are the same as those usually understood by a person skilled in the art. The terminology used in this specification are merely intended to describe specific embodiments, and is not intended to limit the present disclosure. The term “and/or” used herein refers to any combination of one or more of associated items listed.

It should be understood that terms such as “first,” “second,” and “third” are used herein to describe various elements and should not be limited by these terms. These terms are used only to distinguish elements of the same type from each other. For example, a first element may also be referred to as a second element, and vice versa, without affecting the scope of the present disclosure. The use of any and all examples or exemplary phrases (“for example,” “e.g.,” “such as,” etc.) provided in this specification is only intended to better illustrate the embodiments of the present disclosure and is not to limit the scope of the present disclosure unless otherwise required.

The embodiments of the present disclosure may be implemented or practiced by computer hardware, a combination of hardware and software, or computer instructions stored in a non-transitory computer-readable memory. The method may be implemented in computer programs developed using standard programming techniques-including a non-transitory computer-readable storage medium configured with a computer program, where the storage medium so configured causes a computer to operate in a specific and predefined manner-according to the methods and figures described in this specification. Each program may be implemented in a high-level procedural or object-oriented programming language to interface with a computer system. However, the program may be implemented in assembly or machine language, if required. In any case, the language may be compiled or interpreted. Moreover, the program can be executed on a dedicated integrated circuit designed for that purpose.

Furthermore, steps of processes described herein may be executed in any suitable order unless otherwise indicated herein or clearly contradicted by context. The processes described herein (or variations and/or combinations thereof) may be carried out under the control of one or more computer systems configured with executable instructions. This process may be implemented as code (e.g., executable instructions, one or more computer programs, or one or more applications) collectively executed on one or more processors, by hardware, or combinations thereof. The computer program includes several instructions executable by one or more processors.

Furthermore, the method may be implemented in any type of computing platform, including but not limited to personal computers, mini-computers, main-frames, workstations, networked or distributed computing environments, and computer platforms separate, integral to, or in communication with charged particle tools or other imaging devices, and the like. Aspects of the present disclosure may be implemented in machine-readable code stored on a non-transitory storage medium or device, whether removable or integral to the computing platform, such as a hard disc, optical read and/or write storage mediums, Random Access Memory (RAM), Read-Only Memory (ROM), and the like, such that it is readable by a programmable computer, for configuring and operating the computer when the computer reads the storage medium or device to perform the processes described herein. Moreover, the machine-readable code, or portions thereof, may be transmitted over wired or wireless networks. The disclosure of this embodiment includes various types of computer-readable storage media, such as media that contains instructions or programs for implementing the steps described above in conjunction with a microprocessor or other data processor. The present disclosure also includes the computer itself when programmed according to the methods and techniques described herein.

Computer programs can be utilized to process input data to perform the functions described herein and thereby transform the input data to generate output data, which can be stored in a non-volatile memory. The output data may also be applied to one or more output devices, such as a display. In preferred embodiments of the present disclosure, the transformed data corresponds to physical and tangible objects, including generating a particular visual representation of the physical and tangible objects on a display.

The foregoing descriptions are merely embodiments of the present disclosure, but are not intended to limit the present disclosure. As long as the technical effects of the present disclosure are achieved by the same means, any modification, equivalent replacement, or improvement made within the protection scope and principle of the present disclosure shall be considered within the protection scope of the present disclosure. Various modifications and variations to the technical schemes and/or implementations of the present disclosure may be made without departing from the protection scope of the present disclosure.

1. A method for rapidly assessing cerebral visual function based on electroencephalogram (EEG) and fast periodic visual stimulation oddball paradigm, comprising:
presenting several sets of visual stimuli images to a target individual, with each set of visual stimuli images examining a specific visual function; wherein the several sets of visual stimuli images evaluate various cerebral visual functions of the target individual, from low-level to high-level processing; visual stimuli images in the same set comprises a series of base stimuli images and a series of oddball stimuli images; the base and oddball stimuli images are interleaved at specific frequencies to form a stimulus sequence; a base frequency is defined as a presentation frequency of the base stimuli images, and an oddball frequency is defined as a presentation frequency of the oddball stimuli images;
collecting EEG signals of the target individual while presenting the visual stimuli images;
detecting a first and a second signal amplitude of the EEG signals, wherein the first signal amplitude corresponds to the base frequency of the target individual, and the second signal amplitude corresponds to the oddball frequency and harmonics of the target individual; and
comparing the first signal amplitude with a third signal amplitude of a healthy individual in a database to determine a visual processing ability score of the target individual, and comparing the second signal amplitude with a fourth signal amplitude of the healthy individual in the database to determine a visual discrimination ability score of the target individual.

2. The method for rapidly assessing cerebral visual function based on EEG and fast periodic visual stimulation oddball paradigm of claim 1, wherein presenting several sets of visual stimuli images to a target individual for respectively examining different cerebral visual functions comprises:
in presenting a sequence of visual stimuli images in the same set, presenting several base stimuli images and several oddball stimuli images, respectively, according to the base frequency and the oddball frequency.

3. The method for rapidly assessing cerebral visual function based on EEG and fast periodic visual stimulation oddball paradigm of claim 1, wherein comparing the first signal amplitude with a third signal amplitude of a healthy individual in a database to determine a visual processing ability score of the target individual, and comparing the second signal amplitude with a fourth signal amplitude of the healthy individual in the database to determine a visual discrimination ability score of the target individual comprises:
presenting several sets of visual stimuli images to individuals in a healthy control group, where visual stimuli images in the same set include a series of base stimuli images and a series of oddball stimuli images, the base stimuli images and the oddball stimuli images in each set of visual stimuli images are interleaved at specific frequencies to form a stimulus sequence, a base frequency is defined as a presentation frequency of the base stimuli images, and an oddball frequency is defined as a presentation frequency of the oddball stimuli images;
collecting EEG signals of the individuals in the healthy control group while presenting the visual stimuli images;
detecting the third signal amplitude and the fourth signal amplitude of the EEG signals, wherein the third signal amplitude corresponds to the base frequency of the individuals in the healthy control group, and the fourth signal amplitude corresponds to an oddball frequency and harmonics of the individuals in the healthy control group;
determining a Signal-to-Noise Ratio (SNR) of the third signal amplitude and an SNR of the fourth signal amplitude, respectively;
determining the visual processing ability score of the target individual according to a percentile of an SNR of the first signal amplitude of the target individual in the SNR of the third signal amplitude of the healthy control group; and determining the visual discrimination ability score of the target individual based on a percentile of an SNR of the second signal amplitude of the target individual in the SNR of the fourth signal amplitude of the healthy control group.

4. The method for rapidly assessing cerebral visual function based on EEG and fast periodic visual stimulation oddball paradigm of claim 1, wherein several sets of base stimuli images and oddball stimuli images are hierarchically and systematically presented based on EEG and fast periodic visual stimulation oddball paradigm, wherein:
the base stimuli images are different from the oddball stimuli images at low-, middle-, and
at the low-level visual function, the base stimuli images and the oddball stimuli images have different contrasts/luminances, different sizes, different colors, different image orientations, or different spatial frequencies, respectively;
at the middle-level visual function, the base stimuli images and the oddball stimuli images have different image motion states or different shapes, respectively; and
at the high-level visual function, the base stimuli images are randomly scrambled object images, and the oddball stimuli images are intact object images; or the base stimuli images are non-face object images, and the oddball stimuli images are face images; or the base stimuli images are non-tool object images, and the oddball stimuli images are tool images; or the base stimuli images are face images of strangers, and the oddball stimuli images are face images of familiar individuals; or the base stimuli images are face images of strangers, and the oddball stimuli images are self-face images.

5. The method for rapidly assessing cerebral visual function based on EEG and fast periodic visual stimulation oddball paradigm of claim 1, wherein the base frequency is greater than the oddball frequency, and the base frequency is a multiple of the oddball frequency.

6. The method for rapidly assessing cerebral visual function based on EEG and fast periodic visual stimulation oddball paradigm of claim 1, wherein the target individual and the individuals of the healthy control group are classified through training using a machine learning method with the base frequency, the oddball frequency and the harmonic characteristics thereof of the target individual and the individuals of the healthy control group.

7. A computer apparatus, comprising a memory and a processor, wherein the memory is configured for storing at least one program, and the processor is configured to load the at least one program to perform the method for rapidly assessing cerebral visual function based on EEG and fast periodic visual stimulation oddball paradigm of claim 1.

8. A non-transitory computer-readable storage medium, having a processor-executable program stored therein, wherein the processor-executable program, when executed by a processor, causes the processor to perform the method for rapidly assessing cerebral visual function based on EEG and fast periodic visual stimulation oddball paradigm of claim 1.
